(19) United States
(12) Patent Application Publication
(10) Pub. No.: US 2019/0206528 A1
(43) Pub. Date: Jul. 4, 2019

(54) METHOD AND SYSTEM FOR MONITORING A PATIENT

(71) Applicant: Wipro Limited, Bangalore (IN)

(72) Inventor: Kothamangala Anandaiah Shetty Nagaraja, Mulbagal (IN)

(73) Assignee: Wipro Limited

(21) Appl. No.: 15/900,884

(22) Filed: Feb. 21, 2018

(30) Foreign Application Priority Data
Dec. 29, 2017 (IN) 201741047345

(51) Int. Cl. G16H 10/60; G06N 99/00

(52) U.S. Cl. CPC G16H 10/60; G06N 99/005

(57) ABSTRACT

Embodiments of present disclosure discloses method and system for monitoring a patient in a care unit. For the monitoring, initially, a patient data from a monitoring device associated with the patient is retrieved. Bundling of the patient data using a micro-bundling method is performed to obtain corresponding one or more bundle features. Further, nearest neighbour parameter associated with the patient data is determined based on the corresponding one or more bundle features. The patient data is classified to be one of critical data and non-critical data based on one or more nearest neighbour parameters. The critical data and the non-critical data is provided to one or more attendants related to the patient.

### TECHNICAL FIELD

The present subject matter is related in general to healthcare technology, more particularly, but not exclusively to a system and method for monitoring a patient in a care unit using micro-bundling method.

### BACKGROUND

Medical institutes such as hospitals, healthcare centres and super specialty health care facilities include care units which may be operation theatres, intensive care units and critical care intensive units. Such care units may be used to accommodate critically ill patients. The care units may also be extensively used in emergency scenarios such as after-surgery recovery, trauma cases, infectious disease patients, coma survival patients, patients on life support system and so on. The patients in a care unit may be observed for a prolonged duration. In such care units, limited access to visit a patient is allowed due to one or more reasons. The one or more reasons include maintenance of hygiene of the care units, patient condition, care unit cleanliness maintenance and so on. Therefore, attendants of the patients, including family and friends of the patients, may not be continuously updated about condition or well-being of the patient. One or more existing systems disclose to monitor conditions of the patient at real-time and provide the conditions to the attendants dynamically. However, processing of the conditions to classify and understand if the conditions are critical or non-critical is not disclosed in the existing systems. Also, the existing systems do not disclose to classify and prioritize the attendants to provide the conditions. Also, data, indicating the conditions, received for a patient may be raw data and processing of the raw data may limit accuracy of the monitoring. The existing systems do not disclose to improve the accuracy of the data for an efficient monitoring.

The information disclosed in this background of the disclosure section is only for enhancement of understanding of the general background of the invention and should not be taken as an acknowledgement or any form of suggestion that this information forms the prior art already known to a person skilled in the art.

### SUMMARY

In an embodiment, the present disclosure relates to a method for monitoring a patient in a care unit. For the monitoring, initially, a patient data from a monitoring device associated with the patient is retrieved. Bundling of the patient data using a micro-bundling method is performed to obtain one or more bundle features. Further, nearest neighbour parameter associated with the patient data is determined based on the one or more bundle features. The patient data is classified to be one of critical data and non-critical data based on one or more nearest neighbour parameters. The critical data and the non-critical data is provided to one or more attendants related to the patient.

In an embodiment, the present disclosure relates to a patient monitoring system for monitoring a patient in a care unit. The patient monitoring system comprises a processor and a memory communicatively coupled to the processor. The memory stores processor-executable instructions, which, on execution, cause the processor to monitor the patient. For the monitoring, initially, a patient data from a monitoring device associated with the patient is retrieved. Bundling of the patient data using a micro-bundling method is performed to obtain one or more bundle features. Further, nearest neighbour parameter associated with the patient data is determined based on the one or more bundle features. The patient data is classified to be one of critical data and non-critical data based on one or more nearest neighbour parameters. The critical data and the non-critical data is provided to one or more attendants related to the patient.

In an embodiment, the present disclosure relates to a non-transitory computer readable medium including instructions stored thereon that when processed by at least one processor cause a device to perform operations to monitor the patient. For the monitoring, initially, a patient data from a monitoring device associated with the patient is retrieved. Bundling of the patient data using a micro-bundling method is performed to obtain one or more bundle features. Further, nearest neighbour parameter associated with the patient data is determined based on the one or more bundle features. The patient data is classified to be one of critical data and non-critical data based on one or more nearest neighbour parameters. The critical data and the non-critical data is provided to one or more attendants related to the patient.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and regarding the accompanying figures, in which:

FIGS. 1a and 1b illustrate exemplary environments for monitoring a patient in accordance with some embodiments of the present disclosure;

FIG. 2 shows a detailed block diagram of a patient monitoring system for monitoring a patient in accordance with some embodiments of the present disclosure;

FIG. 3 illustrates a flowchart showing an exemplary method for monitoring a patient, in accordance with some embodiments of present disclosure;

FIG. 4 illustrates a flowchart showing an exemplary method for providing critical data and non-critical data to one or more attendants related to a patient in accordance with some embodiments of present disclosure; and

FIG. 5 illustrates a block diagram of an exemplary computer system for implementing embodiments consistent with the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

The terms “includes”, “including”, or any other variations thereof, are intended to cover a non-exclusive inclusion, such that a setup, device or method that includes a list of components or steps does not include only those components or steps but may include other components or steps not expressly listed or inherent to such setup or device or method. In other words, one or more elements in a system or apparatus proceeded by “includes . . . a” does not, without more constraints, preclude the existence of other elements or additional elements in the system or method.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

Medical institutes such as hospitals, healthcare centres, super specialty health care facilities and so on, include care units to accommodate patients in critical conditions. As per regulations of the medical institutes, attendants, including family members and friends of the patients, may be prohibited from entering the care units to visit the patients. The present disclosure provisions a method and system for monitoring a patient in a care unit and providing patient data to the attendants. The present disclosure discloses to classify the patient data to be one of critical data and non-critical data and further classifies each of the attendants to be one of a primary attendant and a secondary attendant. At least one of the primary attendant and the secondary attendant is selected to provide at least one of the critical data and the non-critical data. The present disclosure implements micro-bundling method for bundling the patient data to obtain one or more bundle features. Further, nearest neighbour parameter associated with the patient data are determined based on the one or more bundle features. The nearest neighbour parameter are used to classify the patient data, by which an accuracy of the classification and the monitoring may be achieved.

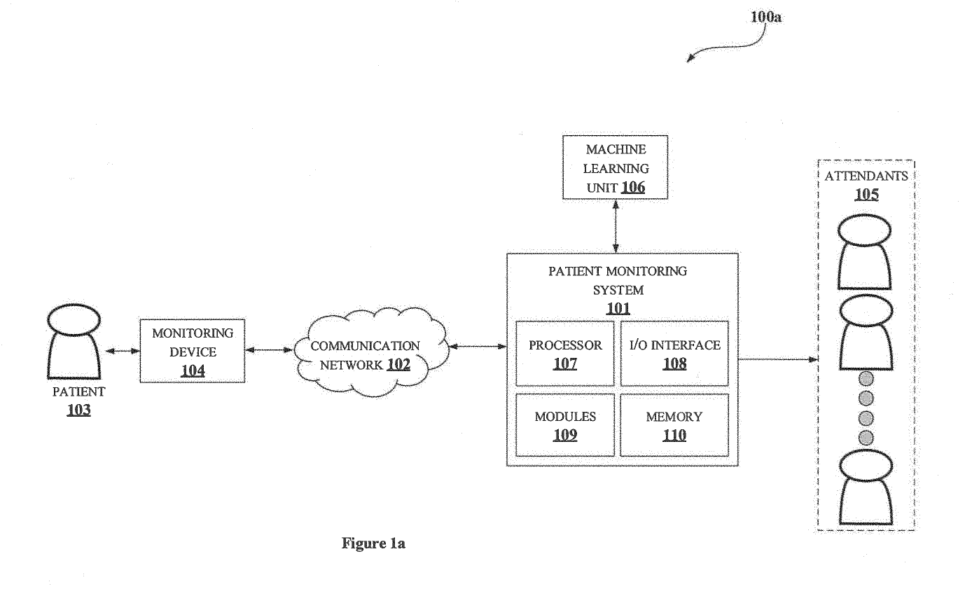

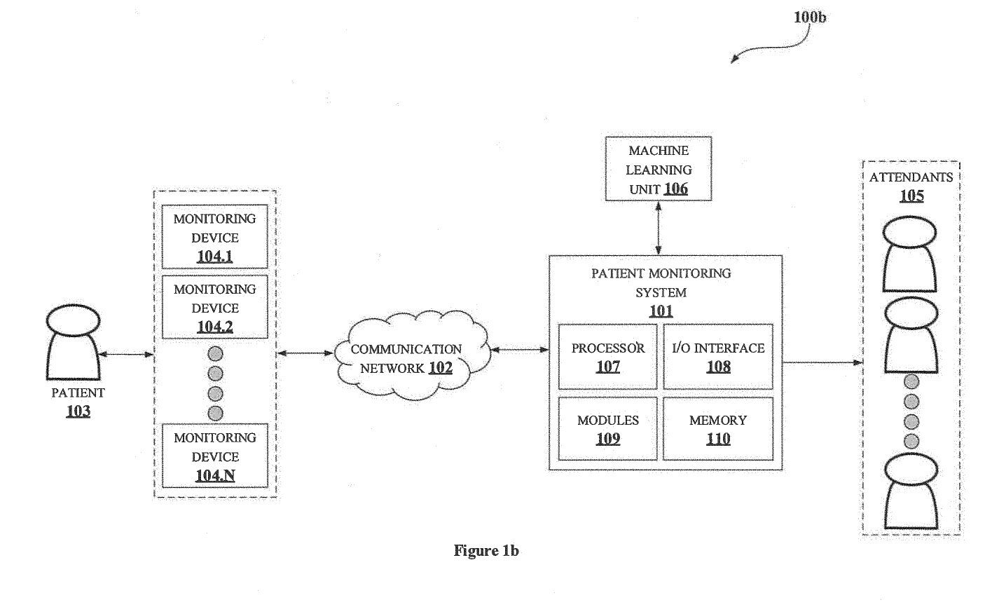

FIGS. 1a and 1b illustrate exemplary environments 100a and 100b of a patient monitoring system 101 for monitoring a patient in a care unit. The exemplary environment 100a comprises the patient monitoring system 101, a communication network 102, a monitoring device 104 associated with a patient 103 and a machine learning unit 106, to monitor and provide patient data to one or more attendants 105. The patient monitoring system 101 may be configured to perform the monitoring of the patient 103 as disclosed in the present disclosure.

The patient monitoring system 101 may communicate with the monitoring device 104 via the communication network 102 as shown in the figure. The monitoring device 104 may be configured to monitor the patient 103 and retrieve the patient data associated with the patient 103. The patient monitoring system 101 may receive the patient data from the monitoring device 104, via the communication network 102. In an embodiment, the patient monitoring system 101 may communicate with the one or more attendants 105 via the communication network 102 (not shown in the figure). The patient monitoring system 101 may communicate with the one or more attendants 105 to provide the patient data to the one or more attendants 105. In an embodiment, the machine learning unit 106 may also communicate with the patient monitoring system 101 via the communication network 102 (not shown in the figure). The patient monitoring system 101 may communicate with the machine learning unit 106 for implementing machine learning in the patient monitoring system 101. In an embodiment, the machine learning unit 106 may be embedded in the patient monitoring system 101 for monitoring the patient 103. In an embodiment, the communication network 102 may include, without limitation, a direct interconnection, Local Area Network (LAN), Wide Area Network (WAN), wireless network (e.g., using Wireless Application Protocol), the Internet, and the like.

Further, the patient monitoring system 101 includes a processor 107, an I/O interface 108, one or more modules 109 and a memory 110. In some embodiments, the memory 110 may be communicatively coupled to the processor 107. The memory 110 stores processor executable instructions, which, on execution, may cause the patient monitoring system 101 to monitor the patient 103 as disclosed in the present disclosure. The patient monitoring system 101 may be implemented in a variety of computing systems, such as a laptop computer, a desktop computer, a Personal Computer (PC), a notebook, a smartphone, a tablet, e-book readers, a server, a network server, and the like.

For the monitoring, initially, a patient data from the monitoring device 104 associated with the patient 103 is retrieved. The patient data comprises one or more vital parameters retrieved from the patient 103 at predefined intervals of time. The vital parameters may include one or more patient parameters retrieved at real-time, disease history of the patient 103, activities of the patient 103 within the care unit. The one or more patient parameters retrieved may include, but not limited to, electrocardiography (ECG), Blood Pressure (BP), oxygen saturation level and so on. The disease history of the patient 103 may include current and previous medical conditions of the patient 103. For example, the patient 103 may have had a heart attack previously or the patient 103 may be a cancer patient. The activities of the patient 103 may include behaviour and actions of the patient 103. For example, the actions of the patient 103 during night time may be retrieved, the behaviour of the patient 103 upon injection of a drug may be retrieved and so on. In an embodiment, one or more monitoring devices 104.1 . . . 104.N may be associated with the patient 103 as shown in FIG. 1b. The patient monitoring system 101 may be configured to communicate with each of the one or more monitoring devices 104.1 . . . 104.N via the communication network 102. The patient monitoring system 101 may retrieve patient data from each of the one or more monitoring devices 104.1 . . . 104.N, for the monitoring.

Upon retrieving the patient data from the monitoring device 104, the patient monitoring system 101 may perform bundling of the patient data using a micro-bundling method. By performing the bundling, one or more bundle features associated with the patient data may be retrieved. In the environment 100b, the bundling may be performed for the patient data received from each of the one or more monitoring devices 104.1 . . . 104.N. By said bundling, the one or more bundling features associated with the patient data from each of the one or more monitoring device 104 may be retrieved. In an embodiment, the one or more bundle features associated with the patient data may comprise at least one of locality data, boundary data, recency data, instances data, class label data, error count data, splitting error threshold data, initial time stamp data and performance threshold data associated with the patient data. In an embodiment, the one or more bundle features of the patient data may be updated based on machine learning technique performed by the machine learning unit 106.

Further, the patient monitoring system 101 determines nearest neighbour parameter associated with the patient data based on the corresponding one or more bundle features. In an embodiment, the nearest neighbour parameter comprises Euclidean distance associated with the patient data and centroid calculated from the one or more bundle features. In an embodiment, the centroid may be calculated from the one or more bundles features. In an embodiment, one or more techniques known to a person skilled in the art may be implemented for calculating the centroid and the Euclidean distance for determining the one or more nearest neighbour parameters.

Further, the patient data is classified to be one of critical data and non-critical data based on the one or more nearest neighbour parameters. The critical data and the non-critical data may be provided to one or more attendants 105 related to the patient 103. The one or more attendants 105 may include family members and friends associated with the patient 103. In an embodiment, the one or more attendants 105 may include caretakers or attendants associated with the patient 103 as well. In an embodiment of the present disclosure, for providing the critical data and the non-critical data, the patient monitoring system 101 may be configured to identify each of the one or more attendants 105 to be one of a primary attendant and a secondary attendant. The identification may be performed using a fuzzy logic method, based on one or more attendant parameters. Further, at least one of the primary attendant and the secondary attendant may be selected to provide at least one of the critical data and the non-critical data. In an embodiment, the selection may also be based on the one or more attendant parameters. The one or more attendant parameters may be data retrieved from electronic medical records of the patient 103 in the medical institute. The one or more attendant parameters may include, but are not limited to, nomination information by the patient 103 during admission process, one or more attendants 105 mentioned in visitor records related to the patient 103, spouse information, parent information and so on. By identifying the primary attendant and the secondary attendant, timely information about the patient 103 in the care unit may be updated to the one or more attendants 105.

In an embodiment, the patient monitoring system 101 may receive data for monitoring the patient 103 through the I/O interface 108 of the patient monitoring system 101. The received data may include, but is not limited to, at least one of the patient data, the one or more attendant parameters, data from the machine learning unit 106 and so on. Also, the patient monitoring system 101 may transmit data for monitoring the patient 103 via the I/O interface 108. The transmitted data may include, but is not limited to, at least one of the critical data, non-critical data, data provided to the machine learning unit 106 for machine learning and so on. The I/O interface 108 may be coupled with the processor 107 of the patient monitoring system 101.

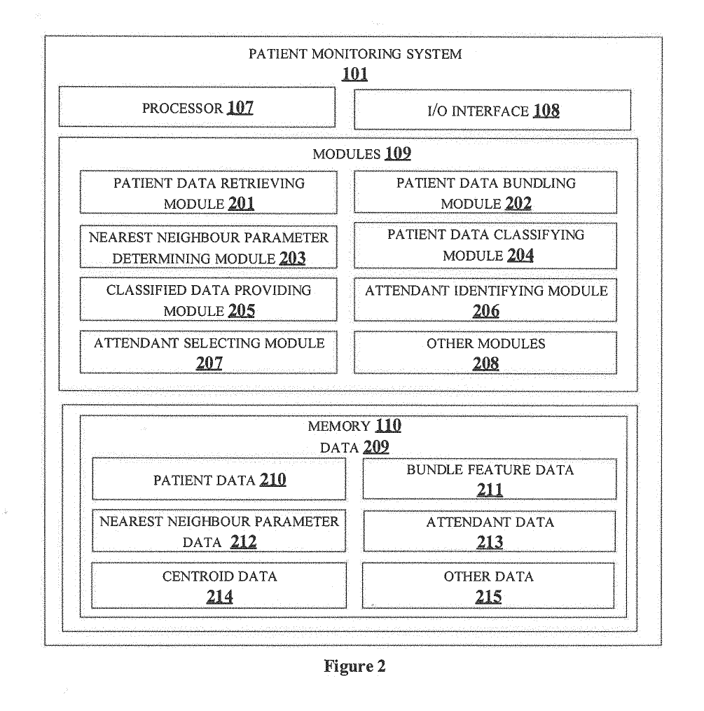

FIG. 2 shows a detailed block diagram of the patient monitoring system 101 for monitoring the patient 103 in accordance with some embodiments of the present disclosure.

The data 209 in the memory 110 and the one or more modules 109 of the patient monitoring system 101 may be described herein in detail.

In one implementation, the one or more modules 109 may include, but are not limited to, a patient data retrieving module 201, a patient data bundling module 202, a nearest neighbour parameter determining module 203, a patient data classifying module 204, a classified data providing module 205, an attendant identifying module 206, an attendant selecting module 207 and one or more other modules 208, associated with the patient monitoring system 101.

In an embodiment, the data 209 in the memory 110 may comprise patient data 210, bundle feature data 211 (also referred as one or more bundle features 211), nearest neighbour parameter data 212 (also referred to nearest neighbour parameter 212), attendant data 213 (also referred to as one or more attendant parameters 213), centroid data 214 (also referred as centroid 214) and other data 215 associated with the patient monitoring system 101.

In an embodiment, the data 209 in the memory 110 may be processed by the one or more modules 109 of the patient monitoring system 101. As used herein, the term module refers to an application specific integrated circuit (ASIC), an electronic circuit, a field-programmable gate arrays (FPGA), Programmable System-on-Chip (PSoC), a combinational logic circuit, and/or other suitable components that provide the described functionality. The one or more modules 109 when configured with the functionality defined in the present disclosure may result in a novel hardware.

For the monitoring, the patient data retrieving module 201 may be configured to retrieve the patient data 210 from the monitoring device 104 associated with the patient 103. The patient data 210 may be automatically monitored and captured by the monitoring device 104 at the predefined intervals of time. In an embodiment, the predefined intervals of time may be provided by doctors and nurses associated with the medical institute. In an embodiment, the monitoring device 104 may include, but is not limited to, at least one of camera, bedside monitor apparatus, vital parameter collection apparatus and so on. In an embodiment, the monitoring device 104 may be associated with Internet of Things (IoT) monitoring devices. The patient data 210 may be retrieved, by the patient data retrieving module 201, dynamically from the patient via the monitoring device 104. In an embodiment, the patient data 210 may be collected by the monitoring device 104 and stored in a memory associated with the monitoring device 104. The patient data retrieving module 201 may be configured to retrieve the stored patient data 210, when required, for monitoring.

In an embodiment, the patient data 210 may be one or more vital parameters which are retrieved based on conditions of the patient 103. The one or more vital parameters may be disease history of the patient 103, activities of the patient 103, electrocardiography (ECG), Blood Pressure (BP), oxygen saturation level and so on. For example, conditions associated with the patient 103 may include abnormal actions of the patient 103 during night, sleeplessness condition of the patient 103 during night, reaction of body of the patient 103 to a drug and so on. Considering such conditions, the patient data 210 may be captured and retrieved at corresponding predefined intervals of time. The predefined intervals of time to retrieve the patient data 210 for the sleeplessness condition of the patient 103 may be at night time. Similarly, the predefined intervals of time to retrieve the patient data 210 for reaction of body of the patient to the drug may be time after injection of the drug. For the patient with heart disease, the patient data 210 may be ECG which may be retrieved continuously. In an embodiment, the patient data 210 retrieved by the patient data retrieving module 201 may be raw data.

Upon retrieving the patient data 210 from the monitoring device 104, the patient data bundling module 202 may perform bundling of the patient data 210 using the micro-bundling method. In an embodiment, the micro-bundling method may also be referred to as the parallel dynamic micro-bundling technique. By performing the bundling, one or more bundle features 211 associated with the patient data 210 may be retrieved. Consider the environment 100b of the patient monitoring system 101. The patient data 210 from each of the one or more monitoring device 104.1 . . . 104.N may be retrieved by the patient data retrieving module 201. The bundling may be performed by the patient data bundling module 202 for the patient data retrieved from each of the one or more monitoring devices 104.1 . . . 104.N. Hence, the one or more bundle features 211 for the patient data 210 associated with each of the one or more monitoring devices 104.1 . . . 104.N may be retrieved. In an embodiment, corresponding one or more bundle features 211 may provide summary of locality of respective patient data 210 received from corresponding monitoring device. In an embodiment, the one or more bundle features 211 associated with the patient data 210 may include, but are not limited to, at least one of locality data, boundary data, recency data, instances data, class label data, error count data, splitting error threshold data, initial time stamp data and performance threshold data associated with the patient data 210. An exemplary representation of the one or more bundle features 211 associated with the patient data 210 is given below:

(BF2x,BF1x,BF2t,BF1t,n) (1)

Bundle feature BF2x may represent sum of squares of patient data attributes and bundle feature BF1x may represent sum of values associated with the patient data 210. Bundle features BF1t and BF2t may represent time stamp data associated with the patient data 210. Bundle feature n may represent number of data instances associated with the patient data 210. In an embodiment, the bundle features BF2x, BF1x, BF1t and BF2t may be in vector form. In an embodiment, the bundle features may be in scalar form. The bundle features BF2x and BF1x may be used to calculate the locality data and boundary data of the patient data 210. The bundle features BF2t and BF1t may be used to determine the recency data of the patient data 210. By using a machine learning technique on the one or more bundle features 211 in equation 1, the one or more bundle features 211 may be updated to one or more bundle features 211 is given below:

(BF2x,BF1x,BF1t,n,BL,ϵ,ts,α,Ω) (2)

From the one or more bundle features 211 as given in equation 2, bundle feature BL represents the class label data, bundle feature ϵ represents the error count data, bundle feature ts represents splitting error threshold data, bundle feature α represents initial time stamp data and bundle feature Ω represents performance threshold data. In an embodiment, the one or more bundle features 211 in equation 2 may be an extended notation of the one or more bundle features 211 in equation 1.

Further, the nearest neighbour parameter determining module 203 determines the nearest neighbour parameter 212 associated with the patient data 210 based on the one or more bundle features 211. In an embodiment, the nearest neighbour parameter 212 comprises Euclidean distance associated with the patient data 210 and the centroid 214 calculated from the one or more bundle features 211. In an embodiment, the centroid 214 may be calculated using equation 3 given below:

$$Centroid = \frac{BF 1 x}{n} \qquad ( 3 )$$

In an embodiment, the Euclidean distance associated with the patient data 210 may be calculated to calculate the centroids of the patient data 210.

For calculating the Euclidean distance, consider data points of the patient data 210 to be (x1, y1) and (x2, y2) in each of one or more bundle features 211. The Euclidean distance may be calculated using equation 4 given below:

Euclidean distance=\sqrt{(x2−x1)²−(y²−y1)²} (4)

In the present disclosure, by calculating the Euclidean distance, the accuracy of the patient monitoring system 101 may be improved. The classification by computing the Euclidean distance decreases computation time associated with the classification, hence the proposed classification works faster when compared to the classification performed using any other distance known to a person skilled in the art. The bundle feature ϵ may be initially 0 and incremented by 1 if the one or more bundle features 211 are used for the classification. Similarly, the bundle feature ϵ may be decremented by 1 if the one or more bundle features 211 are involved in the classification. The bundle feature is may be user defined upper limit of acceptable ϵ from each of the one or more bundle features 211 associated with the patient data 210. The bundle feature is may be maintained at low values to adapt to faster machine learning in the patient monitoring system 101. A larger value of the bundle feature ts may be more tolerant to noise but may not provision faster machine learning. With the increase in number of labelled instances for learning, distribution of the one or more bundle features 211 may vary. The class label data of the nearest one or more bundle features 211 may be assigned to output data instance based on the centroid 214 which may be either one of below and above a threshold value.

Further, in the present disclosure, the centroid data 214 calculated using equation 3 may be used to calculate mean average centroid value associated with the patient data 210. The mean average centroid value may be used to classify the patient data 210 to be one of the critical data and the non-critical data. In an embodiment, the average centroid value may be calculated using equation 5 given below:

$$Mean avearge centroid value = \frac{\begin{matrix}sum of bundle features associated \\ with patient data\end{matrix}}{n} \qquad ( 5 )$$

If the mean average centroid value is greater than the threshold value, then the patient data 210 may be classified to be the critical data by the patient data classifying module 204. If the mean average centroid value is lesser than the threshold value, then the patient data 210 may be classified to be the non-critical data by the patient data classifying module 204. In an embodiment, the threshold value may be associated with the patient data 210 and may be based on, but is not limited to, at least one of the one or more vital parameters, parameters set by the medical institute, patient illness parameters, patient activities and so on.

The critical data and the non-critical data may be provided to one or more attendants 105 related to the patient 103 by the classified data providing module 205. In an embodiment of the present disclosure, for providing the critical data and the non-critical data, the attendant identifying module 206 may be configured to identify each of the one or more attendants 105 to be one of a primary attendant and a secondary attendant. The identification may be performed using a fuzzy logic method, based on one or more attendant parameters 213. Further, at least one of the primary attendant and the secondary attendant may be selected by the attendant selecting module 207 to provide at least one of the critical data and the non-critical data. In an embodiment, the selection may also be based on the one or more attendant parameters 213. The one or more attendant parameters may be data retrieved from electronic medical records of the patient 103 in the medical institute. The one or more attendant parameters may include, not are not limited to, nomination information by the patient 103 during admission process, one or more attendants 105 mentioned in visitor records related to the patient spouse information, parent information and so on. For example, an attendant present during admission of the patient 103 may require knowing the critical data of the patient 103 to arrange funds. Therefore, the attendant may be classified as the primary attendant and the critical data may be provided to the attendant. Similarly, an attendant may be a distant relative of the patient 103 and may not be interested in knowing hospitalization details of the patient 103. Said attendant may be interested in knowing patient condition. Hence, the attendant may be classified as the secondary attendant. By this, the attendant classification module provides hierarchy for sharing the critical data and the non-critical data with the one or more attendants 105.

The other data 215 may store data, including temporary data and temporary files, generated by modules for performing the various functions of the patient monitoring system 101. The one or more modules 109 may also include other modules 208 to perform various miscellaneous functionalities of the patient monitoring system 101. It will be appreciated that such modules may be represented as a single module or a combination of different modules.

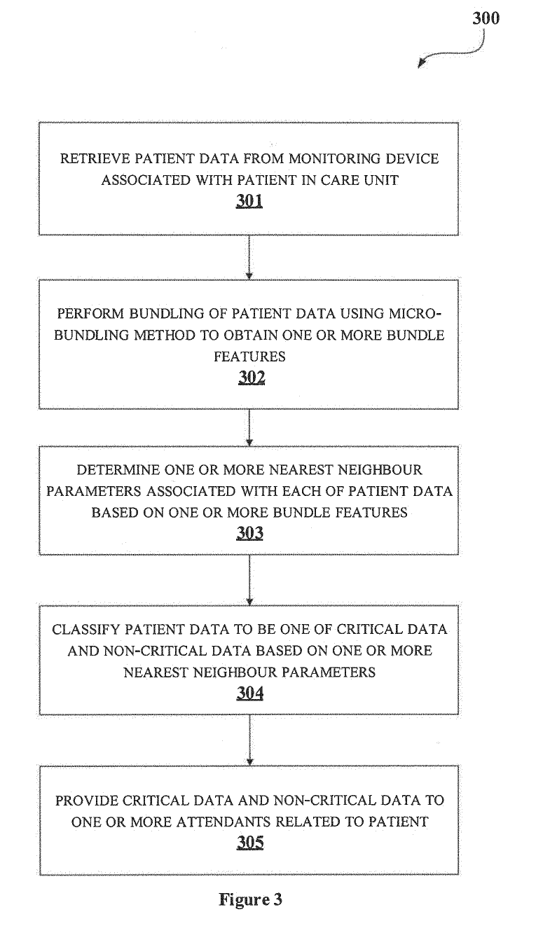

FIG. 3 illustrates a flowchart showing an exemplary method 300 for monitoring the patient 103 in the care unit, in accordance with some embodiments of present disclosure.

At block 301, the patient data retrieving module 201 may retrieve the patient data 210 from the monitoring device 104 associated with the patient 103 in the care unit. The patient data 210 may comprise one or more vital parameters retrieved from the patient at a predefined intervals of time. When the patient monitoring system 101 is associated with one or more monitoring devices 104.1 . . . 104.N, the patient data 210 from each of the one or more monitoring devices 104.1 . . . 104.N may be retrieved by the patient data retrieving module 201.

At block 302, the patient data bundling module 202 may perform bundling of the patient data retrieved at block 301. By performing the bundling, the one or more bundling features associated with the patient data 210 may be retrieved. The micro-bundling method may be implemented for performing the bundling.

At block 303, the nearest neighbour parameter determining module 203 may determine the nearest neighbour parameter 212 associated with the patient data 210 based on the one or more bundle features 211.

At block 304, the patient data classifying module 204 may classify the patient data 210 to be one of the critical data and the non-critical data. The classification may be performed based on the nearest neighbour parameter 212.

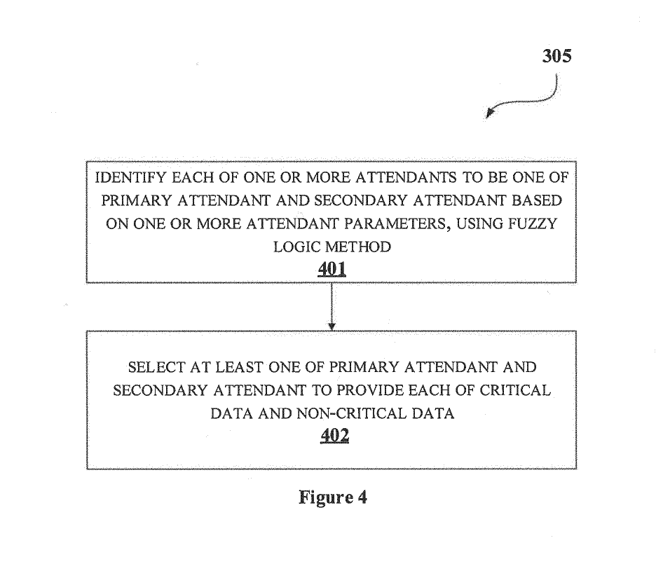

At block 305, the classified data providing module 205 may provide the critical data and the non-critical data to the one or more attendants 105 related to the patient 103. Further, FIG. 4 illustrates a method for providing the critical data and the non-critical data to the one or more attendants 105.

At block 401, the attendant identifying module 206 identifies each of the one or more attendants 105 to be one of the primary attendant and the secondary attendant based on the one or more attendant parameters 213. The identification may be performed using the fuzzy logic method.

At block 402, the attendant selecting module 207 selects at least one of the primary attendant and the secondary attendant to provide at least one of the critical data and the non-critical data.

As illustrated in FIGS. 3 and 4, the methods 300 and 305 may include one or more blocks for executing processes in the patient monitoring system 101. The methods 300 and 305 may be described in the general context of computer executable instructions. Generally, computer executable instructions can include routines, programs, objects, components, data structures, procedures, modules, and functions, which perform particular functions or implement particular abstract data types.

The order in which the methods 300 and 305 are described may not intended to be construed as a limitation, and any number of the described method blocks can be combined in any order to implement the method. Additionally, individual blocks may be deleted from the methods without departing from the scope of the subject matter described herein. Furthermore, the method can be implemented in any suitable hardware, software, firmware, or combination thereof.

### Computing System

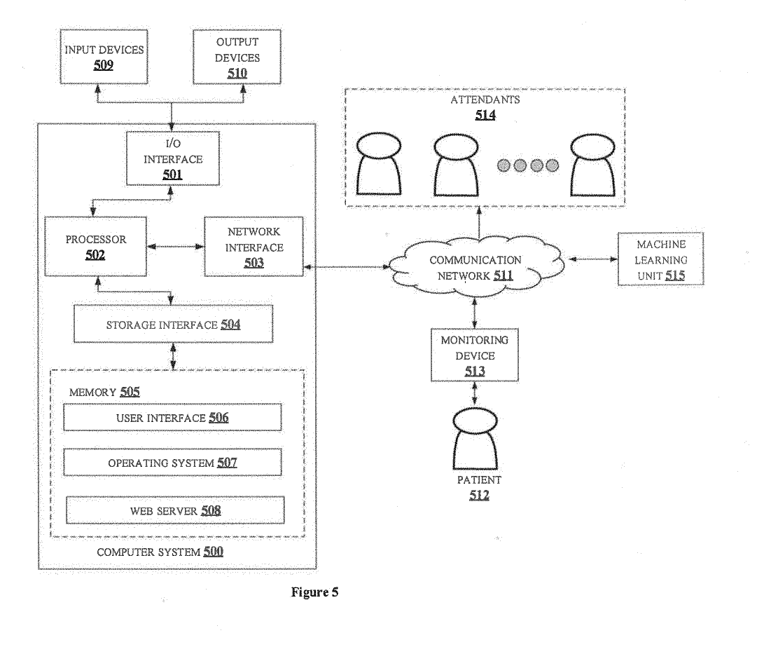

FIG. 5 illustrates a block diagram of an exemplary computer system 500 for implementing embodiments consistent with the present disclosure. In an embodiment, the computer system 500 is used to implement the patient monitoring system 101. The computer system 500 may include a central processing unit (“CPU” or “processor”) 502. The processor 502 may include at least one data processor for executing processes in Virtual Storage Area Network. The processor 502 may include specialized processing units such as, integrated system (bus) controllers, memory management control units, floating point units, graphics processing units, digital signal processing units, etc.

The processor 502 may be disposed in communication with one or more input/output (I/O) devices 509 and 510 via I/O interface 601. The I/O interface 601 may employ communication protocols/methods such as, without limitation, audio, analog, digital, monoaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMax, or the like), etc.

Using the I/O interface 501, the computer system 500 may communicate with one or more I/O devices 509 and 510. For example, the input devices 509 may be an antenna, keyboard, mouse, joystick, (infrared) remote control, camera, card reader, fax machine, dongle, biometric reader, microphone, touch screen, touchpad, trackball, stylus, scanner, storage device, transceiver, video device/source, etc. The output devices 510 may be a printer, fax machine, video display (e.g., cathode ray tube (CRT), liquid crystal display (LCD), light-emitting diode (LED), plasma, Plasma display panel (PDP), Organic light-emitting diode display (OLED) or the like), audio speaker, etc.

In some embodiments, the computer system 500 may consist of the patient monitoring system 101. The processor 502 may be disposed in communication with the communication network 511 via a network interface 503. The network interface 503 may communicate with the communication network 511. The network interface 503 may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, etc. The communication network 511 may include, without limitation, a direct interconnection, local area network (LAN), wide area network (WAN), wireless network (e.g., using Wireless Application Protocol), the Internet, etc. Using the network interface 503 and the communication network 511, the computer system 500 may communicate with the monitoring device 513 to retrieve patient data from the patient 512, one or more attendants 514 and a machine learning unit 515 for monitoring the patient 512. The network interface 503 may employ connection protocols include, but not limited to, direct connect, Ethernet (e.g., twisted pair 10/100/1000 Base T), transmission control protocol/internet protocol (TCP/IP), token ring, IEEE 802.11a/b/g/n/x, etc.

The communication network 511 includes, but is not limited to, a direct interconnection, an e-commerce network, a peer to peer (P2P) network, local area network (LAN), wide area network (WAN), wireless network (e.g., using Wireless Application Protocol), the Internet, Wi-Fi and such. The first network and the second network may either be a dedicated network or a shared network, which represents an association of the different types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the first network and the second network may include a variety of network devices, including routers, bridges, servers, computing devices, storage devices, etc.

In some embodiments, the processor 502 may be disposed in communication with a memory 505 (e.g., RAM, ROM, etc. not shown in FIG. 5) via a storage interface 504. The storage interface 504 may connect to memory 505 including, without limitation, memory drives, removable disc drives, etc., employing connection protocols such as, serial advanced technology attachment (SATA), Integrated Drive Electronics (IDE), IEEE-1394, Universal Serial Bus (USB), fibre channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 505 may store a collection of program or database components, including, without limitation, user interface 506, an operating system 507 etc. In some embodiments, computer system 500 may store user/application data 506, such as, the data, variables, records, etc., as described in this disclosure. Such databases may be implemented as fault-tolerant, relational, scalable, secure databases such as Oracle® or Sybase®.

The operating system 507 may facilitate resource management and operation of the computer system 500. Examples of operating systems include, without limitation, APPLE MACINTOSH® OS X, UNIX®, UNIX-like system distributions (E.G., BERKELEY SOFTWARE DISTRIBUTION™ (BSD), FREEBSD™, NETBSD™, OPENBSD™, etc.), LINUX DISTRIBUTIONS™ (E.G., RED HAT™, UBUNTU™, KUBUNTU™, etc.), IBM™ OS/2, MICROSOFT™ WINDOWS™ (XP™, VISTA™/7/8, 10 etc.), APPLE® IOS™, GOOGLE® ANDROID™, BLACKBERRY® OS, or the like.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include Random Access Memory (RAM), Read-Only Memory (ROM), volatile memory, non-volatile memory, hard drives, CD ROMs, DVDs, flash drives, disks, and any other known physical storage media.

### Advantages

An embodiment of the present disclosure provisions a system for intelligent real-time patient monitoring and data sharing with one or more attendants. By this, the one or more attendants are aware of condition of the patient without frequent visits into care unit. Also, this facilitates cleaner care unit premises.

An embodiment of the present disclosure facilitates in capturing minimum optimal data from the patient data instead of taking complete raw data, for processing, using micro-bundling method. Overall, the present disclosure provisions an efficient approach of reduction of retrieved patient data without any compromise on errors.

An embodiment of the present disclosure provisions minimization of error rate associated with patient data by performing proposed classification and calculation of centroid.

The described operations may be implemented as a method, system or article of manufacture using standard programming and/or engineering techniques to produce software, firmware, hardware, or any combination thereof. The described operations may be implemented as code maintained in a “non-transitory computer readable medium”, where a processor may read and execute the code from the computer readable medium. The processor is at least one of a microprocessor and a processor capable of processing and executing the queries. A non-transitory computer readable medium may include media such as magnetic storage medium (e.g., hard disk drives, floppy disks, tape, etc.), optical storage (CD-ROMs, DVDs, optical disks, etc.), volatile and non-volatile memory devices (e.g., EEPROMs, ROMs, PROMs, RAMs, DRAMs, SRAMs, Flash Memory, firmware, programmable logic, etc.), etc. Further, non-transitory computer-readable media may include all computer-readable media except for a transitory. The code implementing the described operations may further be implemented in hardware logic (e.g., an integrated circuit chip, Programmable Gate Array (PGA), Application Specific Integrated Circuit (ASIC), etc.).

Still further, the code implementing the described operations may be implemented in “transmission signals”, where transmission signals may propagate through space or through a transmission media, such as, an optical fibre, copper wire, etc. The transmission signals in which the code or logic is encoded may further comprise a wireless signal, satellite transmission, radio waves, infrared signals, Bluetooth, etc. The transmission signals in which the code or logic is encoded is capable of being transmitted by a transmitting station and received by a receiving station, where the code or logic encoded in the transmission signal may be decoded and stored in hardware or a non-transitory computer readable medium at the receiving and transmitting stations or devices. An “article of manufacture” includes non-transitory computer readable medium, hardware logic, and/or transmission signals in which code may be implemented. A device in which the code implementing the described embodiments of operations is encoded may include a computer readable medium or hardware logic. Of course, those skilled in the art will recognize that many modifications may be made to this configuration without departing from the scope of the invention, and that the article of manufacture may include suitable information bearing medium known in the art.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary a variety of optional components are described to illustrate the wide variety of possible embodiments of the invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated operations of FIGS. 3 and 4 show certain events occurring in a certain order. In alternative embodiments, certain operations may be performed in a different order, modified or removed. Moreover, steps may be added to the above described logic and still conform to the described embodiments. Further, operations described herein may occur sequentially or certain operations may be processed in parallel. Yet further, operations may be performed by a single processing unit or by distributed processing units.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERRAL NUMERALS

| Reference | |
|---|---|
| Number | Description |
| 100a and 100b | Environment |
| 101 | Patient monitoring system |
| 102 | Communication network |
| 103 | Patient |
| 104 | Monitoring device |
| 104.1 . . . 104.N | One or more monitoring devices |
| 105 | One or more attendants |
| 106 | Machine learning unit |
| 107 | Processor |
| 108 | I/O interface |
| 109 | Modules |
| 110 | Memory |
| 201 | Patient data retrieving module |
| 202 | Patient data bundling module |
| 203 | Nearest neighbour parameter determining module |
| 204 | Patient data classifying module |
| 205 | Classified data providing module |
| 206 | Attendant identifying module |
| 207 | Attendant selecting module |
| 208 | Other modules |
| 209 | Data |
| 210 | Patient data |
| 211 | One or more bundle features |
| 212 | Nearest neighbour parameter |
| 213 | One or more attendant parameters |
| 214 | Centroid data |
| 215 | Other data |
| 500 | Computer System |
| 501 | I/O Interface |
| 502 | Processor |
| 503 | Network Interface |
| 504 | Storage Interface |
| 505 | Memory |
| 506 | User Interface |
| 507 | Operating System |
| 508 | Web Server |
| 509 | Input Devices |
| 510 | Output Devices |
| 511 | Communication Network |
| 512 | Patient |
| 513 | Monitoring device |
| 514 | Attendants |
| 515 | Machine learning unit |

We claim:

1. A method for monitoring a patient, the method comprising:
retrieving, by a patient monitoring system (101), a patient data (210) from a monitoring device associated with a patient (103) in a care unit;
performing, by the patient monitoring system (101), bundling of the patient data (210) using a micro-bundling method, to obtain one or more bundle features (211);
determining, by the patient monitoring system (101), nearest neighbour parameter (212) associated with the patient data (210), based on the one or more bundle features (211);
classifying, by the patient monitoring system (101), the patient data (210) to be one of critical data and non-critical data based on the nearest neighbour parameter (212); and
providing, by the patient monitoring system (101), the critical data and the non-critical data patient data (210) to one or more attendants (105) related to the patient, for monitoring the patient.

2. The method as claimed in claim 1 further comprising:
identifying, by the patient monitoring system (101), each of the one or more attendants (105) to be one of a primary attendant and a secondary attendant, based on one or more attendant parameters (213), using a fuzzy logic method; and
selecting, by the patient monitoring system (101), at least one of the primary attendant and the secondary attendant to provide at least one of the critical data and the non-critical data.

3. The method as claimed in claim 1, wherein the patient data (210) comprises one or more vital parameters retrieved from the patient at a predefined intervals of time.

4. The method as claimed in claim 1, wherein the one or more bundle features (211) of the patient data (210) are updated based on machine leaning technique.

5. The method as claimed in claim 4, wherein the one or more bundle features (211) comprises at least one of locality data, boundary data, recency data, instances data, class label data, error count data, splitting error threshold data, initial time stamp data and performance threshold data, associated with the patient data (210).

6. The method as claimed in claim 1, wherein the nearest neighbour parameter (212) comprises Euclidean distance associated with the patient data (210) and centroid (214) calculated from the one or more bundles features.

7. A patient monitoring system (101) for monitoring a patient (103), the patient monitoring system (101) comprises:
a processor (107); and
a memory communicatively coupled to the processor (107), wherein the memory stores processor-executable instructions, which, on execution, cause the processor (107) to:
retrieve a patient data (210) from a monitoring device (104) associated with a patient (103) in a care unit;
perform bundling of the patient data (210) using a micro-bundling method, to obtain one or more bundle features (211);
determine nearest neighbour parameter (212) associated with the patient data (210), based on the one or more bundle features (211);
classify the patient data (210) to be one of critical data and non-critical data based on the nearest neighbour parameter (212); and
provide the critical data and the non-critical data to one or more attendants related to the patient (103), for monitoring the patient (103).

8. The patient monitoring system (101) as claimed in claim 7 further comprises the processor (107) configured to:
identify each of the one or more attendants to be one of a primary attendant and a secondary attendant, based on one or more attendant parameters (213), using a fuzzy logic method; and
select at least one of the primary attendant and the secondary attendant to provide at least one of the critical data and the non-critical data.

9. The patient monitoring system (101) as claimed in claim 7, wherein the patient data (210) comprises one or more vital parameters retrieved from the patient (103) at a predefined intervals of time.

10. The patient monitoring system (101) as claimed in claim 7, wherein the one or more bundle features (211) of the patient data (210) are updated based on machine leaning technique.

11. The patient monitoring system (101) as claimed in claim 10, wherein the one or more bundle features (211) comprises at least one of locality data, boundary data, recency data, instances data, class label data, error count data, splitting error threshold data, initial time stamp data and performance threshold data, associated with the patient data (210).

12. The patient monitoring system (101) as claimed in claim 7, wherein the nearest neighbour parameter (212) comprises Euclidean distance associated with the patient data (210) and centroid (214) calculated from the one or more bundles features.

13. A non-transitory computer readable medium including instructions stored thereon that when processed by at least one processor cause a device to perform operations comprising:
retrieving a patient data (210) from a monitoring device associated with a patient (103) in a care unit;
performing bundling of the patient data (210) using a micro-bundling method, to obtain one or more bundle features (211);
determining nearest neighbour parameter (212) associated with the patient data (210), based on the one or more bundle features (211);
classifying the patient data (210) to be one of critical data and non-critical data based on the nearest neighbour parameter (212); and
providing the critical data and the non-critical data patient data (210) to one or more attendants (105) related to the patient, for monitoring the patient.

14. The medium as claimed in claim 13 further comprising:
identifying, by the patient monitoring system (101), each of the one or more attendants (105) to be one of a primary attendant and a secondary attendant, based on one or more attendant parameters (213), using a fuzzy logic method; and
selecting, by the patient monitoring system (101), at least one of the primary attendant and the secondary attendant to provide at least one of the critical data and the non-critical data.

15. The medium as claimed in claim 13, wherein the patient data (210) comprises one or more vital parameters retrieved from the patient at a predefined intervals of time.

16. The medium as claimed in claim 13, wherein the one or more bundle features (211) of the patient data (210) are updated based on machine leaning technique.

17. The medium as claimed in claim 16, wherein the one or more bundle features (211) comprises at least one of locality data, boundary data, recency data, instances data, class label data, error count data, splitting error threshold data, initial time stamp data and performance threshold data, associated with the patient data (210).

18. The medium as claimed in claim 13, wherein the nearest neighbour parameter (212) comprises Euclidean distance associated with the patient data (210) and centroid (214) calculated from the one or more bundles features.
